(19) United States
(12) Patent Application Publication
(10) Pub. No.: US 2016/0213318 A1
(43) Pub. Date: Jul. 28, 2016

(54) SENSORS FOR DETECTING ACUTE STROKE AND METHOD OF USING SAME

(71) Applicants: Juan Parodi, San Isidro Pcia. Buenos Aires (AR); Ramon Berguer, West Bloomfield, MI (US)

(72) Inventors: Juan Parodi, Buenos Aires (AR); Ramon Berguer, West Bloomfield, MI (US)

(21) Appl. No.: 15/008,084

(22) Filed: Jan. 27, 2016

Related U.S. Application Data
(60) Provisional application No. 62/107,110, filed on Jan. 23, 2015.

(51) Int. Cl. A61B 5/00; A61B 5/0492; A61B 5/04; A41D 19/00

(52) U.S. Cl. CPC A61B 5/6806; A41D 19/0027; A61B 5/0492; A61B 5/4064; A61B 5/746; A61B 5/0024; A61B 5/04012

(57) ABSTRACT

A system and method for detecting stroke in an individual, and in particular a sleeping individual. The individual has sensors on a least one hand or wrist for detecting electrical and/or muscular activity. The sensors may be included in or on a glove or bracelet worn by the individual. The absence of electrical and/or muscular activity is indicative of a stroke, and when such absence is detected, an alert is raised. Absence of detecting electrical and/or muscular activity can be detected in only one hand and/or wrist to avoid false alarms from REM sleep which results in the absence of electrical and/or muscular activity in both hands.

### FIELD OF THE INVENTION

Embodiments of the invention relate to sensors, for example wearable sensors, for detecting acute stroke, and methods of using the sensors.

### BACKGROUND OF THE INVENTION

Approximately 15.3 million strokes occur annually worldwide and about one third are fatal. Stroke is the second leading cause of death and accounts for significant disability, institutionalization, and health care cost. Strokes increase exponentially with advanced age, and, of course, the population ages. Strokes occur more frequently in African Americans, Native Americans and elderly women.

Risk factors for stroke include carotid disease, hypertension, atrial fibrillation, diabetes, smoking and sleep apnea. Men with moderate-to-severe sleep apnea had an almost threefold increased risk of ischemic stroke. Obstructive sleep apnea is among the most common chronic disorders in adults, occurring in 4% of middle-aged men and 2% of middle-aged women.

In the last decade, treatment of acute ischemic stroke caused by embolization from a carotid plaque or from atrial fibrillation has improved dramatically as a result of the use of local lytic agents and mechanical thrombectomy. These methods have allowed the recovery by patients that would previously have had a bad prognosis.

Time is often of the essence when attempting to reperfuse the brain tissue threatened by i schemia. In general, the opportunity to reverse a stroke exists within 3 hours of its occurrence. Today, patients can often be treated within three hours of the onset of the stroke and the success rate of this timely intervention is high. However, when the stroke occurs while the patient sleeps, it is likely that, by the time it is discovered, the patient cannot be treated until well after this 3 hour window of opportunity.

About one third of ischemic strokes occur during sleep. Embodiments of the present invention propose to solve this problem by allowing for detection of a stroke during sleep, thereby permitting immediate treatment.

There are at least 5 million patients in the United States with atrial fibrillation, which carries with it a 1 in 4 risk of cerebral emboli during the lifetime. Patients with severe carotid stenosis, patent foramen ovale, carotid dissections and shaggy aortas are also prone to develop ischemic cerebral emboli. Embodiments of the present invention can be particularly helpful for these high-risk patients.

### BRIEF DESCRIPTION OF THE DRAWINGS

FIG. 1 shows a dorsum side of a glove according to an embodiment described herein;

FIG. 2 shows the palm side of the glove of FIG. 1;

FIG. 3A shows the palm side of a glove according to another embodiment described herein;

FIG. 3B shows the dorsum side of the glove of FIG. 3A;

FIG. 4 shows the inner portion of a bracelet according to embodiments described herein;

FIG. 5 shows the outer portion of a the bracelet of FIG. 4;

FIG. 6 shows a system arranged according to embodiments described herein; and

FIG. 7 shows a method of detecting a stroke according to embodiments described herein.

### DETAILED DESCRIPTION OF THE INVENTION

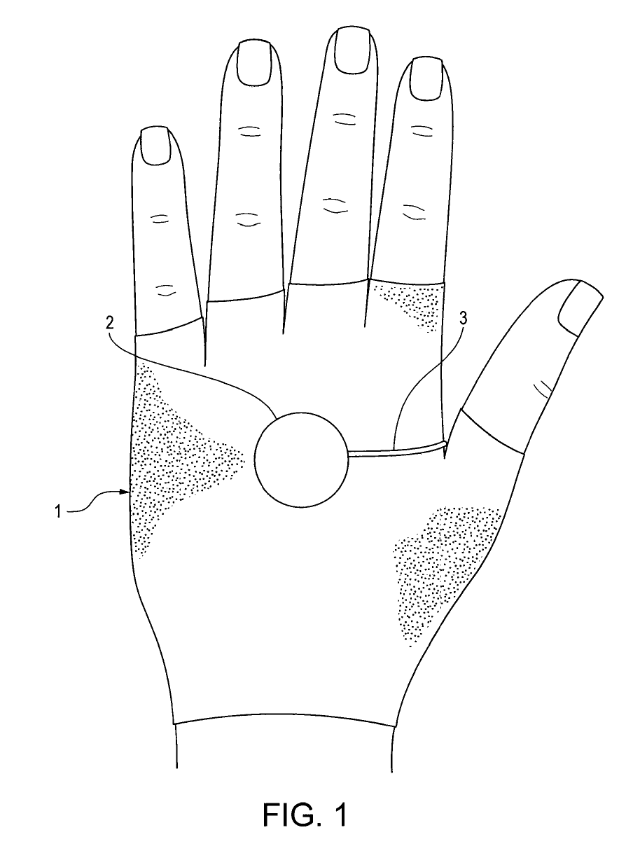

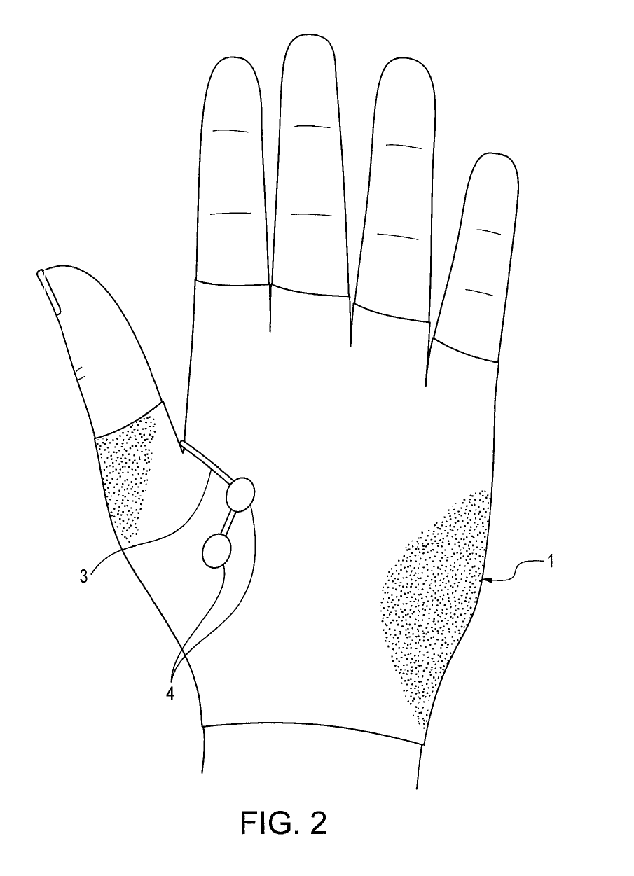

Unilateral arm paralysis is the most common manifestation of stroke. When a stroke occurs during sleep, there is cessation of the spontaneous and repeated electrical activity of muscles that occurs at frequent intervals. An object of embodiments of the present invention is to detect this cessation of electrical and muscle activity that takes place as soon as a stroke occurs and implement an automatic alarm system that will permit a curative intervention. This can be accomplished by detecting the absence of this periodic electrical and muscular activity transcutaneously by means of, for example, electromyography (EMG) electrodes. In one embodiment shown in FIGS. 1 and 2, the cessation of electrical and muscle activity can be detected in the area of the hand that has the most musculature (the thenar eminence at the base of the thumb). In this embodiment, the electromyography electrodes (e.g., sensors 4 of FIGS. 1 and 2) will be attached to a glove 1 (e.g., the inner surface) to keep the electrodes 4 in position.

FIGS. 1 and 2 show an example of such a glove 1 according to an embodiment or the invention. FIG. 1 shows a view of the dorsum of a hand wearing the glove 1, and FIG. 2 shows a view of the palm of the hand wearing the glove 1. Both of these figures show the glove 1 on a left hand, but a mirror image of glove 1 can instead be worn on the right hand.

In the embodiment of FIGS. 1 and 2, the glove 1 includes two electromyography sensors 4. The sensors 4 (FIG. 2) in the embodiment of FIGS. 1 and 2 are located on (either the inside or outside) or in the glove 1 so that they sit in the thenar eminence region of the hand, but the sensors could be placed in a different location on the glove 1. Sensors 4 are two of the three EMG sensor electrodes. The third EMG sensor electrode can be placed in any appropriate place (e.g., dorsum). While the embodiment of FIGS. 1 and 2 shows two sensors, embodiments of the present invention contemplate any number of sensors. Each sensor 4 detects electrical and/or muscle activity in the hand.

The sensors 4 are connected to a transmitter 2 (FIG. 1) by cable 3. In a preferred embodiment shown in FIG. 1, the transmitter 2 is located on (either the inside or outside) or in the glove 1 such that it is on the dorsum of the hand. The transmitter 2 receives signals from the sensors 4 via cable 3 and transmits those signals. The transmitter 2 can use any wireless protocol for transmission, for example Bluetooth®.

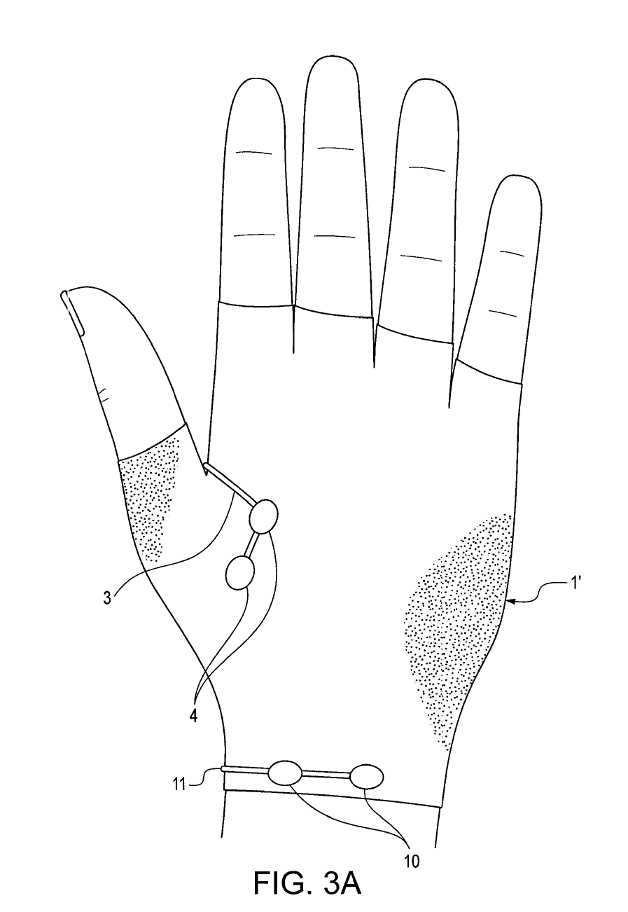

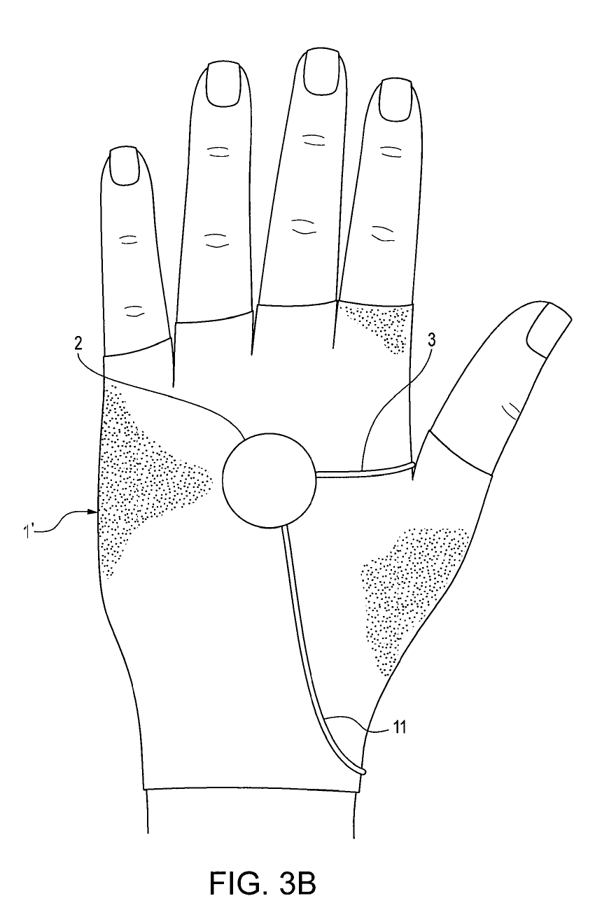

FIGS. 3a and 3b show an embodiment of a glove 1′ that is different from glove 1 in FIGS. 1 and 2 in that it also includes sensors 10 arranged on the wrist. Sensors 10 can be the same type of sensors as sensors 4, and are used to sense electrical and muscular activity in the wrist. Sensors 10 are connected to transmitter 2 by cable 11.

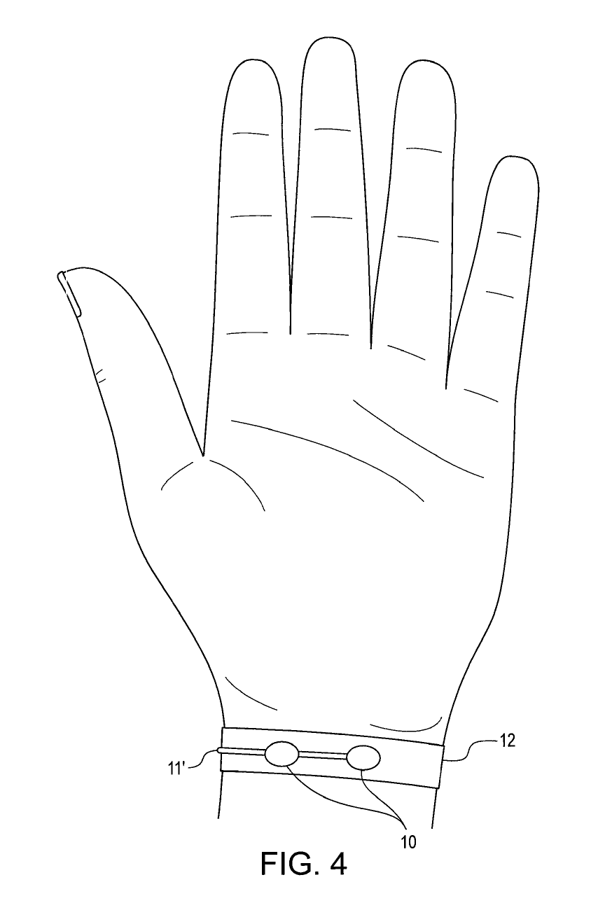

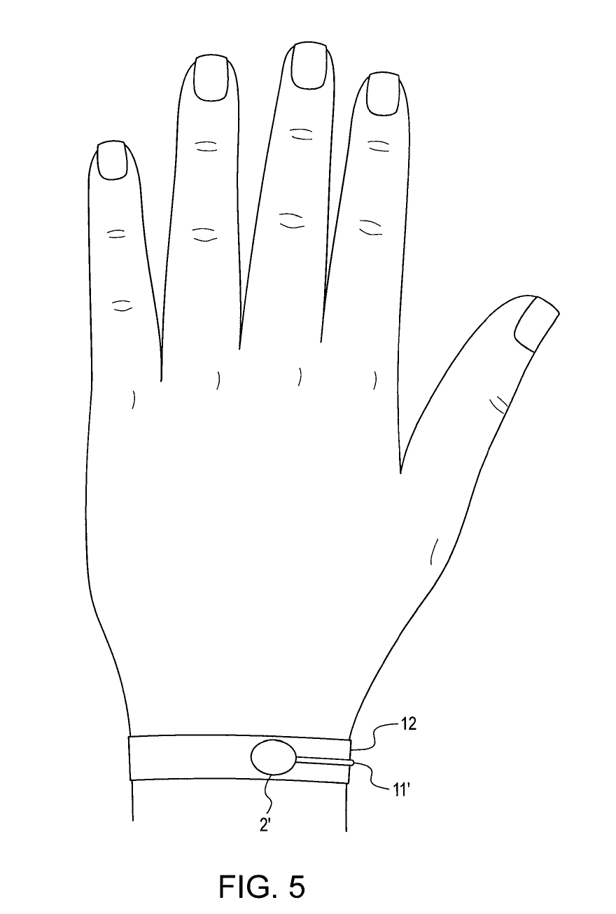

FIGS. 4 and 5 show an embodiment of a bracelet 12 containing only sensors 10 on the wrist. Sensors 10 are connected to transmitter 2′ by cable 11′.

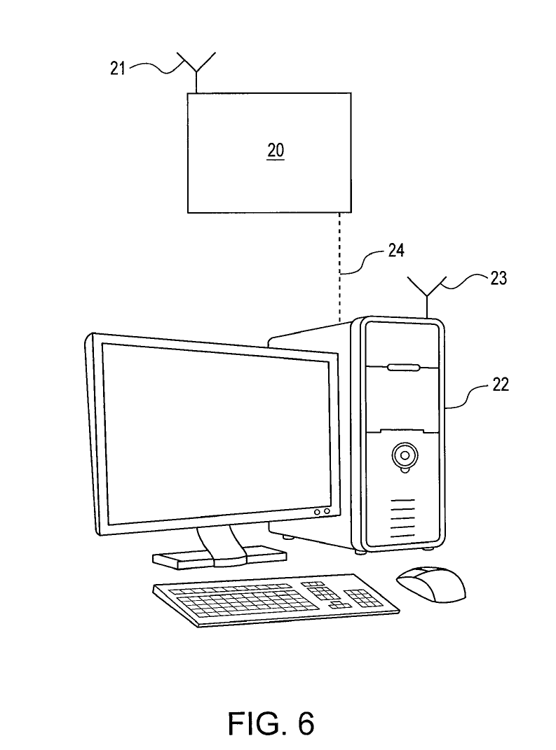

FIG. 6 shows a portion of the system according to embodiments of the invention that receives the signal from the transmitters 2, 2′ described in FIGS. 1-5. The transmitter wirelessly transmits the signals received from the sensors (e.g, sensors 4) to a microcontroller 20 (FIG. 6) at the bedside. The microcontroller 20 may process the signals (e.g., analog-to-digital conversion and rectification) and is configured to identify EMG signals. The microcontroller provides the processed EMG signals to a computing device 22, for example a desktop or laptop computer, smartphone, tablet or any other type of computing device.

The microcontroller 20 can send the processed EMG signals to computing device 22 wirelessly using receiver/transmitter 21 and receiver/transmitter 23. This wireless transmission can be any type of wireless transmission, including wife or Bluetooth®. Alternatively, the microcontroller 20 can send the processed signals to computing device 22 by cable 24.

The computing device 22 is configured to analyze (e.g., by a software program) the EMG signals to determine the presence of a stroke. In the embodiment described above, if the computing device 22 determines that the EMG signals show an absence of electrical or muscular activity for an established period of time, a stroke is detected and the computing device 22 can automatically initiate an alarm system. For example, it can sound an audible alarm by, for example, placing a phone call to the patient's home. The microcontroller can also, or alternatively, alert emergency services.

Intervention within the three-hour window significantly increases the probability of recovery. Within this window, the earlier the patient is brought to the interventional suite, the lower the risk of intracerebral bleeding during rescue.

A typical night's sleep includes approximately four to five periods of what is called rapid eye movement (REM) when dreams occur. This REM typically comprises 20-25% of total sleep time in adults (about 90-120 minutes). During REM, brain activity is similar to the brain activity that occurs while awake, but there is paralysis of muscular activity that prevents movement during dreams.

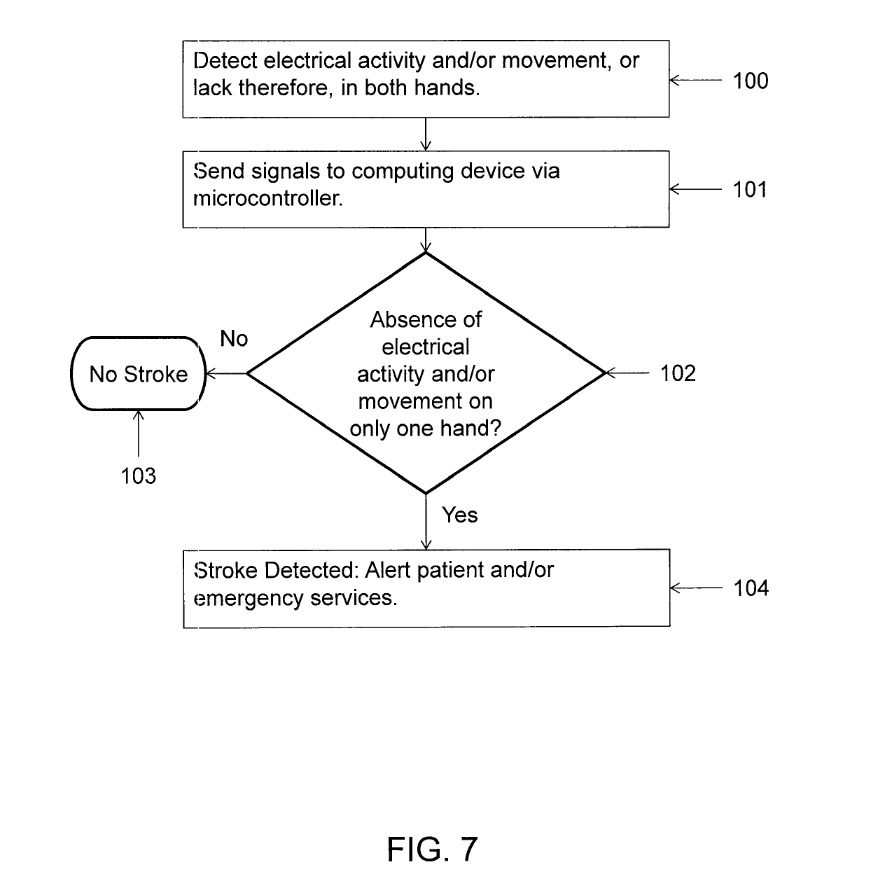

An embodiment of the present invention provides a mechanism to distinguish the absence of signals representing electrical and muscle activity caused by REM from that caused by a stroke. In this embodiment, described below with respect to FIG. 7, a glove and/or bracelet according to the above-described embodiments is worn on both hands and/or wrists to detect the absence or presence of electrical and/or muscular activity in the hands/wrists (step 100, FIG. 7). The detected signals are sent from the gloves/bracelets on each hand/wrist to the microcontroller 20 (FIG. 6) for processing. The microcontroller sends processed signals to the computing device 22 of FIG. 6 (step 101, FIG. 7). The computing device 22 then determines if there is an absence of electrical and/or muscular activity on one hand/wrist, but not on the other (step 102, FIG. 7). If the computing device 22 determines that sensors (4 and/or 10) in the gloves and/or bracelets detect absence of electrical and muscle activity on both hands and/or wrists, REM sleep, instead of a stroke, is detected and the sensors will not trigger the alarm (step 103, FIG. 7). If electrical or muscular activity is detected in both hands/wrists, this also means that no stroke is detected (step 103, FIG. 7). If absence of electrical and muscle activity is only detected on one hand, a stroke has been detected and the computing device 22 will trigger the alarm (step 104, FIG. 7).

While the embodiment of FIGS. 1-5 shows wireless communication between the glove and a microcontroller, the glove can also send signals via a wire.

What is claimed as new and desired to be protected by Letters Patent of the United States is:

1. A method of detecting a stroke in a sleeping individual, the method comprising:
detecting on a right hand and a left hand signals representing the presence or absence of at least one of electrical activity and muscular activity;
determining that the individual is having a stroke if the absence of electrical activity and muscular activity is detected in only one hand or is not detected in either hand; and
determining that the individual is not having a stroke if the absence of electrical activity and muscular activity is detected in both the right hand and the left hand.

2. The method of claim 1, further comprising:
alerting the individual if it is determined that the individual is having a stroke.

3. The method of claim 2, wherein the alerting step comprises placing a call to the individual's telephone.

4. The method of claim 1, further comprising alerting emergency services if it is determined that the individual is having a stroke.

5. The method of claim 1, wherein the detecting step is performed on the thenar eminence region of each hand.

6. The method of claim 1, further comprising:
sending the signals from each hand to a microcontroller.

7. The method of claim 6, wherein the sending step is performed via Bluetooth®.

8. The method of claim 6, further comprising:
processing the signals at the microcontroller and then sending the processed signals to a computing device; and
performing the determining steps at the computing device.

10. The method of claim 1, wherein the detecting step is performed by sensors in or on a glove worn on each hand.

11. A system for detecting stroke in a sleeping individual, the system comprising:
a right-hand sensor and a left-hand sensor, each sensor for detecting signals representing at least one of electrical activity and muscular activity in the respective hand;
a transmitter for transmitting the signal to a microcontroller;
a computing device configured to receive the signal from the microcontroller and determine that the individual is having a stroke if an absence of electrical activity and muscular activity is detected in only one of the right-hand sensor and the left-hand sensor, and to determine that the individual is not having a stroke if an absence of electrical activity and muscular activity is detected in both the right-hand sensor and the left-hand sensor.

12. The system of claim 11, further comprising:
a right glove worn on the right hand of the individual and a left glove worn on the left hand of the individual, the right glove having the right-hand sensor and the left glove having the left-hand sensor.

13. The system of claim 12, wherein the respective sensor is arranged in or on the right glove and left glove such that each sensor is placed on the thenar eminence region of the hand.

14. The system of claim 11, wherein the computing device is further configured to raise an alert when it is determined that the individual is having a stroke.

15. The system of claim 14, wherein the alert comprises alerting emergency services.

16. The system of claim 14, wherein the alert comprises placing a call to the telephone of the individual.

17. The system of claim 11, wherein the right-hand sensor and left-hand sensor each comprise a plurality of sensors.

18. The system of claim 11, wherein the transmitter is a Bluetooth® transmitter.

19. A glove worn on a hand for detecting a stroke in an individual, the glove comprising:
at least one sensor for sensing signals representing at least one of electrical activity and muscular activity in the hand;
a transmitter for receiving the signals and wirelessly transmitting the signals to a device capable of raising an alert when a stroke is detected.

20. The glove of claim 19, wherein the at least one sensor is arranged in or on the glove such that it will be placed on the thenar eminence of the hand.

21. The glove of claim 19, wherein the at least one sensor is an electromyography electrode.

22. The glove of claim 19, further comprising at least one second sensor arranged on the glove such that it will be placed on the wrist of the individual, the at least one second sensor for sensing signals representing at least one of electrical activity and muscular activity in the wrist and configured to provide the signals to the transmitter.
